(19) United States
(12) Patent Application Publication
(10) Pub. No.: US 2011/0297160 A1
(43) Pub. Date: Dec. 8, 2011

(54) PIVOTABLE ENDOTRACHEAL TUBE ASSEMBLIES

(76) Inventor: Richard Farah, Eagle River, AK (US)

(21) Appl. No.: 13/149,689

(22) Filed: May 31, 2011

Related U.S. Application Data
(60) Provisional application No. 61/352,488, filed on Jun. 8, 2010.

(51) Int. Cl. A61M 16/04

(52) U.S. Cl. 128/207.15; 128/207.14

(57) ABSTRACT

A pivotable endotracheal tube assembly includes an insertion tube defining a first ventilating lumen and adapted for insertion in the trachea of a patient, a connection tube defining a second ventilating lumen, and a pivotable fitting adapted to interconnect the tubes to thereby communicate the first and second ventilating lumens and provide free pivotal movement of one relative to the other. The pivotable fitting may include a a distal portion coupled to the insertion tube and having a female component, and may further include a proximal portion coupled to the connection tube and having a male component coaxially telescoped at least partially within the female component. A radial flange on the male component configured to be axially slidable within a corresponding groove in the female component may provide continuous and relative pivotable movement of the components.

### RELATED APPLICATION

This application claims the benefit of U.S. Provisional Application No. 61/352,488 filed on Jun. 8, 2010, the entire disclosure of which is incorporated herein by reference for all purposes.

### TECHNICAL FIELD

The disclosure relates to endotracheal (“ET”) tubes, and in particular to ET tube assemblies that include a pivotable fitting.

### BACKGROUND

Endotracheal (“ET”) tubes are used to couple a medical patient's respiratory system to a breathing apparatus during medical procedures, such as in surgical or emergency situations, and so forth. A typical ET tube consists of a length of somewhat flexible tubing, such as made from polyvinyl chloride or the like, with an inflatable cuff positioned around the distal end that is advanced into the patient's trachea, generally through the patient's mouth. The ET tube defines a ventilating lumen that can be sealed relative to the trachea by inflating the cuff, generally through an inflation line that extends from the cuff toward the proximal end of the tubing. When inflated, the cuff prevents or at least restrains movement of the distal end of the ET tube by friction against the tracheal wall. The proximal portion of the tubing extends from the patient's mouth, and a fitting on the proximal end thereof couples the ET tube to suitable apparatus, such as an artificial respirator or ventilator.

Primarily because standard ET tubes are of unitary construction, they are not satisfactory for the range of applications in which they are employed. For example, during a medical procedure, it may be necessary to reposition or otherwise move the proximal end of the ET tube and/or attached tubing that connects the ET tube to the ventilating apparatus, so that medical personnel may properly provide medical care. If not purposely repositioned, the proximal end of the ET tube and/or attached tubing may be inadvertently bumped or otherwise moved during medical care. However, movement of the proximal end usually results in corresponding movement of the intubated end, which may stress or even damage the intubated patient's tracheal tissues. In addition, movement of the proximal end of the ET tube relative to the intubated, distal end may twist or bind the tubing, restricting or interrupting ventilation. On the other hand, proper repositioning an ET tube is a complex and time-consuming procedure (requiring cuff deflation, careful adjustment of the EY tube position, and then cuff reinflation). However, medical attention is often time-sensitive and may not allow time for proper repositioning. Moreover, even if done properly, repositioning may place strain on the intubated patient's tracheal tissues.

### SUMMARY

Illustrative embodiments of a pivotable endotracheal tube assembly are disclosed herein to include an insertion tube consisting of a first tubular member defining a first ventilating lumen therethrough and having a distal portion adapted for insertion in the trachea of a patient, a connection tube consisting of a second tubular member defining a second ventilating lumen therethrough, and a pivotable fitting adapted to interconnect the proximal ends of the insertion tube and the connection tube to thereby communicate the first and second ventilating lumens and provide free pivotal movement of the connection tube relative to the insertion tube. Some embodiments may further include an inflatable cuff coaxial with and secured to the distal portion of the insertion tube and a cuff inflation tube extending longitudinally from the cuff toward the proximal end of the insertion tube.

In some embodiments, the pivotable fitting provides a singe degree of freedom of movement and includes a fitting body adapted to interconnect the insertion tube at an angle relative to the connection tube. In such embodiments, the fitting body may include pivotally interconnected and coaxial proximal and distal portions, with the distal portion defining a distal port adapted to couple with the insertion tube, and a proximal portion having a side arm adapted to couple with the connection tube, wherein the side arm is disposed at the angle relative to the pivot axis. In some embodiments, the fitting body includes a male component (on one of the proximal and distal portions) coaxially telescoped at least partially within, and adapted for free rotation relative to, a female component (on the other of the proximal and distal portions), for example by means of a radial flange on the distal end of the male component which is axially slidable within a corresponding groove in the proximal and of the female component. In such embodiments, the male component may be continuously pivotable relative to the female component, or the components may include additional structure or be otherwise configured such that the male component is pivotably indexable through a range of discrete positions relative to the female component.

In some embodiments, a compound pivotable fitting provides two degrees of freedom of movement. Such embodiments may include a fitting body that includes proximal and distal portions pivotally interconnected along a first pivot axis, with the distal portion defining a distal port adapted to couple with the insertion tube, and a proximal portion having a side arm disposed at an angle relative to the first pivot axis, with the side arm including proximal and distal side arm portions pivotally interconnected along a second pivot axis, with the distal side arm portion having a second side arm adapted to couple with the connection tube, wherein the second side arm is disposed at a second predetermined angle relative to the second pivot axis.

The concepts and components listed above are clarified with reference to the accompanying drawings and detailed description below.

### BRIEF DESCRIPTION OF THE DRAWINGS

FIG. 1 shows an elevation view of a first embodiment of a pivotable endotracheal tube assembly constructed in accordance with the present disclosure and consisting of an insertion tube and a connection tube interconnected via a pivotable fitting.

FIG. 2 shows the pivotable fitting of the pivotable endotracheal tube assembly of FIG. 1 shown in cross-section, in which a male component is partially telescoped within a female component, and in which a radial flange of the male component is slidably received in a corresponding groove in the female component.

FIG. 3 shows a cross-sectional view of the radial flange and corresponding groove, respectively, of the male and female components, along the line 3-3 of FIG. 1.

FIG. 4 shows a cross-sectional view, similar to that in FIG. 3, of an alternative configuration of a radial flange and corresponding groove, respectively, of male and female components of a pivotable fitting constructed in accordance with the present disclosure.

FIG. 5 shows a top view of the pivotable endotracheal tube assembly of FIG. 1 with the cap removed, and illustrating how the male component of the pivotable, fitting is pivotable relative to the female component.

FIG. 6 shows a partial elevation view of a second embodiment of a pivotable endotracheal tube assembly constructed in accordance with the present disclosure and consisting of an insertion tube and a connection tube interconnected via a compound pivotable fitting.

### DETAILED DESCRIPTION

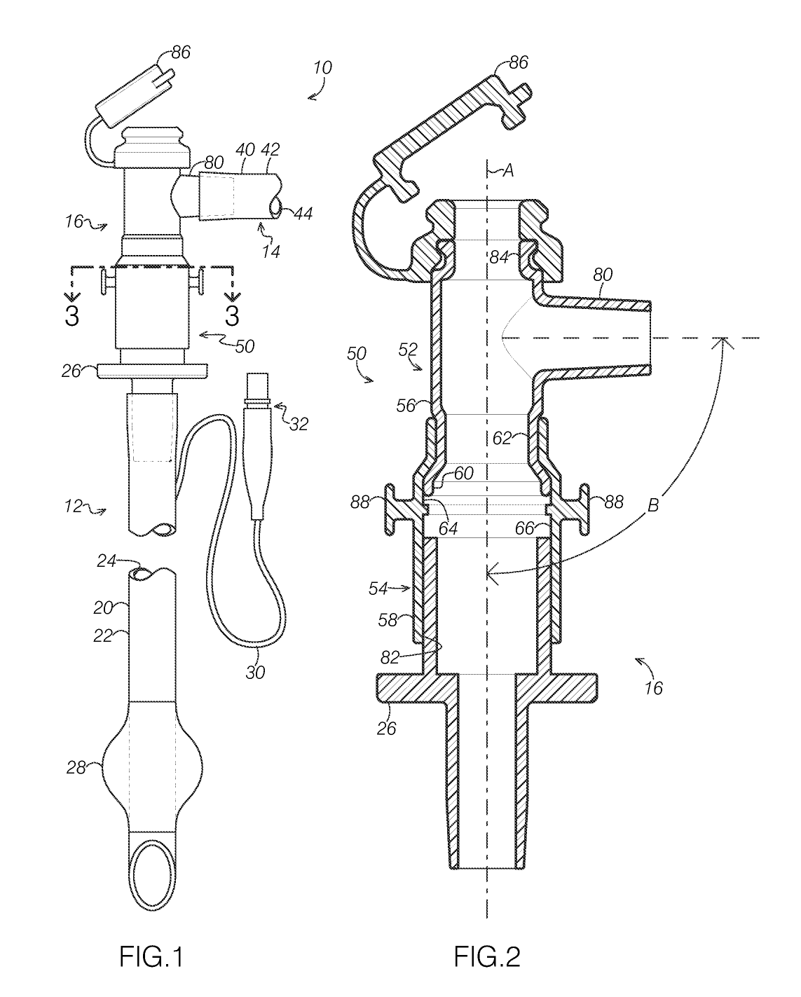

Referring to the drawings, a first embodiment of an endotracheal tube assembly 10 is shown to include an insertion tube 12 and a connection tube 14 interconnected by a pivotable fitting 16. Insertion tube 12 consists generally of a first tubular member 20, shown as a length of flexible medical tubing 22, that defines a first ventilating lumen 24 therethrough. As known to those skilled in the medical arts, the length, curve, flex, diameter, and other characteristics of the tubular member are chosen to suit a particular application and/or patient (e.g., infants will generally require a smaller and/or shorter tube than adults). As such, although not required to all embodiments, the ET tube assembly 10 in FIG. 1 is shown to incorporate a standard or universal connector 26 to interconnect the pivotable fitting 16 with the proximal end of the insertion tube 12, such as via a tapered nipple adapted to slide within and frictionally retain the tubular member. Other embodiments may optionally include different structure interposed between the pivotable fitting and the insertion tube, or a direct connection between the pivotable fitting and the insertion tube, and still others may include a configuration in which the pivotable fitting and the insertion tube are of unitary construction.

The distal portion of insertion tube 12 is adapted for insertion into the trachea of a patient and as such is shown to include at its distal end an inflatable cuff 28 that is secured to and surrounds a portion of the exterior surface of the tubular member. Cuff 28, which is generally formed of a light-gauge latex material or the like, is shown in FIG. 1 in an inflated state, and communicates with a cuff inflation tube 30, the distal portion of which is integral with the wall of the tubular member. The proximal portion is shown to be configured, such as by means of connector 32, for connection to a suitable pump, or the like.

Connection tube 14 consists generally of a second tubular member 40, also shown as a length of flexible medical tubing 42 that defines a second ventilating lumen 44 therethrough. The proximal end of connection tube 14 is shown to be directly connected with the pivotable fitting 16, with the distal end (not shown) being suitably configured to couple with ventilating or other medical apparatus. As above with respect to the insertion tube, other embodiments may include connecting structure interposed between the pivotable fitting and the connection tube, or may include a configuration in which the pivotable fitting and the connection tube are of unitary construction, and so forth.

The pivotable fitting 16 is adapted to interconnect the proximal ends of the insertion and connection tubes, Being hollow, the pivotable fitting communicates the first and second ventilating lumens of the lengths of tubing interconnected thereby. Further, the pivotable fitting, which is shown in greater detail in FIG. 2, provides free pivotal movement of the connection tube relative to the insertion tube. As such, by providing at least one degree of freedom of movement between the connection and insertion tubes, the fitting 16 enables adjustment or movement of one portion of the ET tube assembly 10, such as the connection tube 14, relative to another portion, such as the insertion tube 12, without undue stress or strain on the patient's tracheal tissues, and without requiring careful repositioning of the intubated portion of the ET tube assembly, such as would be the case with a standard, non-pivotable ET tube.

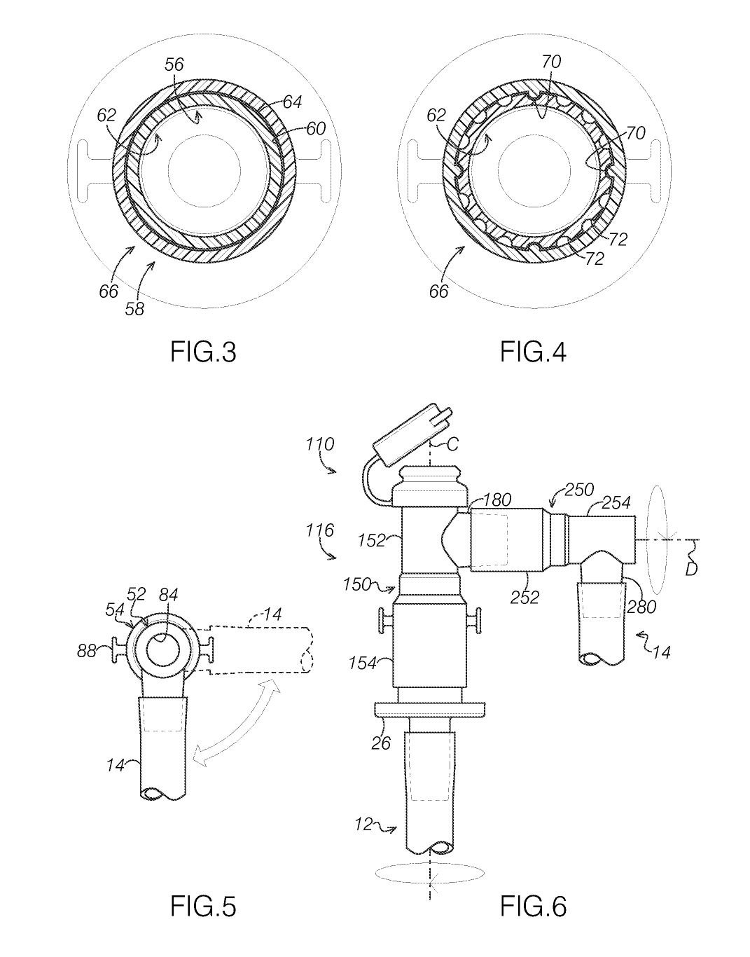

As shown in FIG. 2, the pivotable fitting 16 includes a fitting body, generally indicated at 50 which includes hollow, generally cylindrical proximal and distal portions 52, 54, which are coaxially interconnected for relative pivotable movement about a pivot axis A. More particularly, in the illustrative embodiment shown in FIGS. 1-2, the proximal portion includes a male component 56 telescoped at least partially within a female component 58 of the distal portion. The pivotal movement is provided by means of an outwardly extending radial flange 60 formed by the cylindrical wall 62 of the distal end of the male component that is received in and cooperates with a corresponding axial groove 64 formed in the cylindrical wall 66 of the proximal end of the female component. Typically, the components of the pivotable fitting are constructed of a rigid plastic with a low friction coefficient and/or with suitable tolerances to provide smooth rotation when the flange slides within the groove, For example, FIG. 3 shows the interface between the cylindrical walls 62, 66 of the male and female components forming the flange and corresponding groove, in which the interfacing surfaces are shown to be smooth, and either directly abutting or slightly spaced from each other, thus allowing the male component to freely and continuously rotate about pivot axis A relative to the female component.

However, in other configurations that include a similar pivotable interface, the materials and/or relative configuration of the components may be chosen to provide a preferred degree of resistance to rotation, and/or to control the manner of rotation. As an example of the latter, FIG. 4 shows an alternative configuration, in which the cylindrical wall 66 of the female component 58 includes a plurality of inwardly-extending nubs 70 and in which the cylindrical wall 62 of the male component 56 is provided with a corresponding array of detents 72 in which the nubs may be received, such that rotation of one of the components relative to the other indexes the fitting through a range of discrete rotational positions corresponding to the nub and detent pattern. Other configurations may optionally include stops or other internal structure to provide a partial rotational range, a range of predetermined relative rotational positions, and so forth. Also, it will be apparent to an artisan that the male and female relationship may be reversed relative to the distal and proximal portions, and further that any suitable pivoting mechanism may be used to enable relative rotation of two or more portions of the pivotable fitting 16, such as a first portion to which the connection tube 14 is connected and a second portion to which the insertion tube 12 is connected.

Although not required to all embodiments, the fitting body 50 is shown in FIGS. 1-2 to interconnect the insertion tube at a predetermined angle B relative to the connection tube. More particularly, the fitting body 50 is shown to include a side arm 80 extending from the proximal portion 52 (to which the connection tube may be attached, such as via a nipple, in a manner similar to that of the standard connector), and a distal port 82 formed in the distal portion 54 (in which is received the standard connector 26, to which the insertion tube may be attached). The distal port is coaxial with the pivot axis A, and the side arm 80 extends at a right angle relative thereto. Of course, in other embodiments, the fitting body may assume a configuration to provide a desired predetermined connection angle.

Although not required to all embodiments, the fitting body shown in FIGS. 1-2 further includes a proximal port 84 that is coaxially aligned with the distal port 82. As will be evident to those skilled in the art, the proximal port may allow medical equipment (such as an intubating stylet, a suctioning catheter, and so forth) to be advanced into the ET tube, or an anesthetic or other fluid to be supplied therethrough, without interrupting or detaching the ventilation circuit. As shown in FIG. 5, in the illustrated embodiment, the proximal port stays aligned with the distal port irrespective of the rotational position of the side arm and connection tube. As shown in FIGS. 1-2, the pivotable fitting is shown to include a detachable cap 86 that may be used to selectively seal the proximal port, such as when the proximal port is not being used. Finally, the fitting body 50 is shown to include a pair of finger grips 88 that protrude radially from opposite sides of the outer surface of the female component 58, for example to facilitate coupling and uncoupling the pivotable fitting 16 relative to one or both of the tubes 12, 14 and/or the connector 26.

As will be evident to the artisan, many variations to the configuration of the pivotable fitting and/or components thereof are possible and are considered to be within the scope of this disclosure. For example, the connection angle (shown as angle B in FIG. 2) at which the connection and insertion tubes are interconnected may be adjustable instead of predetermined, such as by including suitable structure. As a non-limiting example of one such variation, FIG. 6 shows a partial view of a second embodiment of an endotracheal tube assembly 110 that features a compound pivotable fitting 116, which provides means to allow this angle to be adjusted, and which also provides two degrees of freedom of movement between the insertion and connection tubes. Briefly, compound pivotable fitting 116 includes a first fitting body 150 having proximal and distal portions 152, 154 pivotally interconnected along a first pivot axis C, with the first proximal portion including a first side arm 180 disposed at right angles thereto. The side arm 180 is shown to be received within a second fitting body 250 that further includes proximal and distal side arm portions 252, 254, which are pivotally interconnected along a second pivot axis D. The distal side arm portion 254 includes a second side arm 280 disposed at right angles thereto. The compound pivotable fitting is thus adapted to interconnect an insertion tube and a connection tube in the same manner as provided by pivotable fitting 10 as shown in FIGS. 1-2; that is, with an insertion tube 12 coupled via a standard connector 26 to a distal port 182 formed in the distal portion 154 of the first fitting body 150, and with a connection tube 14 coupled to the second side arm 280 of the distal side arm portion 254 of the second fitting body 250. As shown, the tubes are oriented in parallel, but in practice, it is contemplated that medical personnel will place the connection tube at a suitable orientation to facilitate medical care to an intubated patient. One or both pivotable connections may be achieved such as via the “flange-in-groove configuration” described above and shown in FIG. 2, such that the various components may be relatively pivoted as shown in FIG. 6, or may incorporate a different manner of pivotable connection. Of course, other alternative configurations of a pivotable fitting may provide for an adjustable connection angle in an alternative manner, such as with one or more joints that include pivots, bearings, ball and socket connections, hinges, and so forth.

Although the present invention has been shown and described with reference to the foregoing operational principles and illustrated examples and embodiments, it will be apparent to those skilled in the art that various changes in form and detail may be made without departing from the spirit and scope of the invention. The present invention is intended to embrace all such alternatives, modifications and variances that fall within the scope of the appended claims.

I claim:

1. A pivotable endotracheal tube assembly comprising:
an insertion tube consisting of a first tubular member defining a first ventilating lumen therethrough and having a distal portion adapted for insertion in the trachea of a patient;
an inflatable cuff coaxial with and secured to the distal portion of the insertion tube;
a cuff inflation tube extending longitudinally from the cuff toward the proximal end of the insertion tube;
a connection tube consisting of a second tubular member defining a second ventilating lumen therethrough; and
a pivotable fitting adapted to interconnect the proximal ends of the insertion tube and the connection tube to thereby communicate the first and second ventilating lumens and provide free pivotal movement of the connection tube relative to the insertion tube.

2. The pivotable endotracheal tube assembly of claim 2, wherein the pivotable fitting includes a fitting body adapted to interconnect the insertion tube at a predetermined angle relative to the connection tube.

3. The pivotable endotracheal tube assembly of claim 2, wherein the fitting body includes pivotally interconnected and coaxial proximal and distal portions, with the distal portion defining a distal port adapted to couple with the insertion tube, and a proximal portion having a side arm adapted to couple with the connection tube, wherein the side arm is disposed at the predetermined angle relative to the pivot axis.

4. The pivotable endotracheal tube assembly of claim 3, wherein the proximal portion further includes a proximal port aligned with the distal port, and wherein the pivotable fitting further includes a cap adapted to selectively seal the proximal port.

5. The pivotable endotracheal tube assembly of claim 4, wherein the distal and proximal ports remain aligned when the distal and proximal portions are pivoted relative to each other.

6. The pivotable endotracheal tube assembly of claim 3, wherein the predetermined angle is a right angle.

7. The pivotable endotracheal tube assembly of claim 1, wherein the pivotable fitting includes a fitting body comprising a male component coaxially telescoped at least partially within, and adapted for free rotation relative to, a female component.

8. The pivotable endotracheal tube assembly of claim 7, wherein the male component includes a distal end telescoped at least partially within a proximal end of the female component, and wherein the distal end of the male component includes a radial flange axially slidable within a corresponding groove in the proximal end of the female component.

9. The pivotable endotracheal tube assembly of claim 7, wherein one of the male and female components s adapted to couple with the tubular member of the connection tube, and the other of the male and female components is adapted to couple with the tubular member of the insertion tube.

10. The pivotable endotracheal tube assembly of claim 9, wherein the proximal end of the male member includes a nipple adapted to slide within and frictionally retain the proximal end of the tubular member of either the connection tube or the insertion tube.

11. The pivotable endotracheal tube assembly of claim 9, wherein the distal end of the female member includes a distal port adapted to accept and frictionally retain the proximal end of a standard connector having a nipple on its distal end that is, in turn, adapted to slide within and frictionally retain the proximal end of the tubular member of either the connection tube or the insertion tube.

12. The pivotable endotracheal tube assembly of claim 7 wherein the fitting body includes one or more finger grips adapted to facilitate coupling and uncoupling the pivotable fitting relative to one or both of the insertion and connection tubes.

13. The pivotable endotracheal tube assembly of claim 12, wherein two finger grips protrude radially from the outer surface of the female component.

14. The pivotable endotracheal tube assembly of claim 7, wherein the male component is continuously pivotable relative to the female component.

15. The pivotable endotracheal tube assembly of claim 7, wherein the male component is pivotably indexable through a range of discrete positions relative to the female component.

16. The pivotable endotracheal tube assembly of claim 1, wherein the pivotable fitting includes a fitting body adapted to interconnect the insertion tube at an adjustable angle relative to the connection tube.

17. The pivotable endotracheal tube assembly of claim 16, wherein the fitting body includes proximal and distal portions pivotally interconnected along a first pivot axis, with the distal portion defining a distal port adapted to couple with the insertion tube, and a proximal portion having a side arm disposed at a predetermined angle relative to the first pivot axis;
and wherein the side arm includes proximal and distal side arm portions pivotally interconnected along a second pivot axis, with the distal side arm portion having a second side arm adapted to couple with the connection tube, wherein the second side arm is disposed at a second predetermined angle relative to the second pivot axis.

18. A pivotable endotracheal tube assembly comprising:
an insertion tube consisting of a first tubular member defining a first ventilating lumen therethrough and having a distal portion adapted for insertion in the trachea of a patient;
a connection tube consisting of a second tubular remember defining a second ventilating lumen therethrough; and
a pivotable fitting having a fitting body that includes proximal and distal portions pivotally interconnected along a pivot axis, the proximal portion having a side arm extending at an angle relative to the pivot axis and the distal portion having a distal port, the side arm and the distal port being respectively adapted to interconnect the proximal ends of the insertion tube and the connection tube, to thereby communicate the first and second ventilating lumens and provide free pivotal movement of the connection tube relative to the insertion tube.

19. The pivotable endotracheal tube assembly of claim 18, wherein the proximal portion further includes a proximal port that remains aligned with the distal port upon pivoting the distal and proximal portions relative to each other, and wherein the pivotable fitting further includes a cap adapted to selectively seal the proximal port.

20. The pivotable endotracheal tube assembly of claim 18, wherein the fitting body comprises a male component coaxially telescoped at least partially within, and adapted for free rotation relative to, a female component.
